(19) United States
(12) Patent Application Publication
(10) Pub. No.: US 2022/0362221 A1
(43) Pub. Date: Nov. 17, 2022

(54) COMBINATION PHARMACOLOGICAL INTERVENTIONS FOR MULTIPLE MECHANISMS OF OBSTRUCTIVE SLEEP APNEA

(71) Applicants: APNIMED, INC. (DELAWARE), Cambridge, MA (US); THE BRIGHAM AND WOMEN'S HOSPITAL, INC., Boston, MA (US)

(72) Inventors: Lawrence G. MILLER, Cambridge, MA (US); Ronald FARKAS, Clarksville, MD (US); D. Andrew WELLMAN, Wayland, MA (US); Luigi TARANTO-MONTEMURRO, Brookline, MA (US); Scott Aaron SANDS, Cambridge, MA (US)

(21) Appl. No.: 17/773,871

(22) PCT Filed: Nov. 3, 2020
(86) PCT No.: PCT/US2020/058714

§ 371 Date: May 3, 2022

Related U.S. Application Data
(60) Provisional application No. 62/930,294, filed on Nov. 4, 2019.

(51) Int. Cl. A61K 31/433; A61K 31/138; A61K 31/216; A61K 9/00; A61P 11/00

(52) U.S. Cl. CPC A61K 31/433; A61K 31/138; A61K 31/216; A61K 9/0053; A61P 11/00

(57) ABSTRACT

In general, the invention relates to pharmaceutical compositions comprising a norepinephrine reuptake inhibitor (NRI), muscarinic receptor antagonist, and carbonic anhydrase inhibitor and methods of treating Sleep Apnea comprising the administration of these pharmaceutical compositions.

### CROSS-REFERENCE TO RELATED APPLICATION

This application claims priority to U.S. Provisional Patent Application No. 62/930,294, filed Nov. 4, 2019, the entire contents of which are incorporated herein by reference.

### TECHNICAL FIELD

The present invention provides pharmaceutical compositions and corresponding methods for lowering loop gain and increasing muscle responsiveness in a subject having Sleep Apnea. The pharmaceutical compositions comprise a norepinephrine reuptake inhibitor (NRI), a muscarinic receptor antagonist (MRA), and a carbonic anhydrase inhibitor (CAI).

### BACKGROUND

Obstructive Sleep Apnea (OSA) is a common disorder caused by collapse of the pharyngeal airway during sleep. OSA can have serious health consequences.

### SUMMARY

One aspect of the present invention provides a method of treating a subject having a condition associated with pharyngeal airway collapse, the method comprising administering to a subject in need thereof an effective amount of (i) a norepinephrine reuptake inhibitor (NRI); (ii) a muscarinic receptor antagonist; and (iii) a carbonic anhydrase inhibitor.

Embodiments of this aspect of the invention may include one or more of the following optional features. In some embodiments, the carbonic anhydrase inhibitor is selected from the group consisting of acetazolamide, dichlorophenamide, dorzolamide, brinzolamide, methazolamide, zonisamide, ethoxzolamide, topiramate, sultiame, and any combinations thereof. In some embodiments, the carbonic anhydrase inhibitor is acetazolamide. In some embodiments, the NRI is selected from the group consisting of Atomoxetine and Reboxetine. In some embodiments, the NRI is Atomoxetine. In some embodiments, the NRI is a norepinephrine selective reuptake inhibitor (NSRI). In some embodiments, the NSRI is selected from the group consisting of Amedalin, Atomoxetine, CP-39,332, Daledalin, Edivoxetine, Esreboxetine, Lortalamine, Nisoxetine, Reboxetine, Talopram, Talsupram, Tandamine, and Viloxazine. In some embodiments, the NRI is a norepinephrine non-selective reuptake inhibitor (NNRI) selected from the group consisting of Amitriptiline, Amoxapine, Bupropion, Ciclazindol, Desipramine, Desvenlafaxine, Dexmethilphenidate, Diethylpropion, Doxepin, Duloxetine, Imipramine, Levomilnacipran, Manifaxine, Maprotiline, Methylphenidate, Milnacipran, Nefazodone, Nortriptyline, Phendimetrazine, Protryptyline, Radafaxine, Tapentadol, Teniloxazine, and Venlafaxine. In some embodiments, the muscarinic receptor antagonist comprises oxybutynin. In some embodiments, the oxybutynin is a substantially pure (R)-oxybutynin. In some embodiments, the oxybutynin is a racemic mixture of (R)-oxybutynin and (S)-oxybutynin. In some embodiments, the carbonic anhydrase inhibitor is administered at a dosage of from about 250 mg to about 750 mg. In some embodiments, the carbonic anhydrase inhibitor is administered at a dosage of about 500 mg. In some embodiments, the NRI is administered at a dosage of from about 20 to about 150 mg. In some embodiments, the NRI is administered at a dosage of from about 50 to about 100 mg. In some embodiments, the muscarinic receptor antagonist ((R)-oxybutynin) is administered at a dosage of from about 1 to about 15 mg. In some embodiments, the muscarinic receptor antagonist ((R)-oxybutynin) is administered at a dosage of from about 2.5 to about 7.5 mg. In some embodiments, the condition associated with pharyngeal airway collapse is Sleep Apnea or Simple Snoring. In some embodiments, the condition associated with pharyngeal airway collapse is Obstructive Sleep Apnea (OSA). In some embodiments, the subject is in a non-fully conscious state. In some embodiments, the non-fully conscious state is sleep. In some embodiments, the NRI, muscarinic receptor antagonist, and carbonic anhydrase inhibitor are administered in a single composition. In some embodiments, the single composition is an oral administration form. In some embodiments, the oral administration form is a syrup, pill, tablet, troche, capsule, or patch.

Another aspect of the invention provides a pharmaceutical composition comprising (i) a norepinephrine reuptake inhibitor (NRI); (ii) a muscarinic receptor antagonist; and (iii) a carbonic anhydrase inhibitor, in a pharmaceutically acceptable carrier.

Embodiments of this aspect of the invention may include one or more of the following optional features. In some embodiments, the carbonic anhydrase inhibitor is selected from the group consisting of acetazolamide, dichlorophenamide, dorzolamide, brinzolamide, methazolamide, zonisamide, ethoxzolamide, topiramate, sultiame, and any combinations thereof. In some embodiments, the carbonic anhydrase inhibitor is acetazolamide. In some embodiments, the NRI is selected from the group consisting of Atomoxetine and Reboxetine. In some embodiments, the NRI is Atomoxetine. In some embodiments, the NRI is a norepinephrine selective reuptake inhibitor (NSRI). In some embodiments, the NSRI is selected from the group consisting of Amedalin, Atomoxetine, CP-39,332, Daledalin, Edivoxetine, Esreboxetine, Lortalamine, Nisoxetine, Reboxetine, Talopram, Talsupram, Tandamine, and Viloxazine. In some embodiments, the NRI is a norepinephrine non-selective reuptake inhibitor (NNRI) selected from the group consisting of Amitriptiline, Amoxapine, Bupropion, Ciclazindol, Desipramine, Desvenlafaxine, Dexmethilphenidate, Diethylpropion, Doxepin, Duloxetine, Imipramine, Levomilnacipran, Manifaxine, Maprotiline, Methylphenidate, Milnacipran, Nefazodone, Nortriptyline, Phendimetrazine, Protryptyline, Radafaxine, Tapentadol, Teniloxazine, and Venlafaxine. In some embodiments, the muscarinic receptor antagonist comprises oxybutynin. In some embodiments, the oxybutynin is a substantially pure (R)-oxybutynin. In some embodiments, the oxybutynin is a racemic mixture of (R)-oxybutynin and (S)-oxybutynin. In some embodiments, the carbonic anhydrase inhibitor is administered at a dosage of from about 250 mg to about 750 mg. In some embodiments, the carbonic anhydrase inhibitor is administered at a dosage of about 500 mg. In some embodiments, the NRI is administered at a dosage of from about 20 to about 150 mg. In some embodiments, the NRI is administered at a dosage of from about 50 to about 100 mg. In some embodiments, the pharmaceutical formulation is in an immediate release formulation. In some embodiments, the pharmaceutical formulation is in an extended release formulation. In some embodiments, the muscarinic receptor antagonist is administered at a dosage of from about 1 to about 15 mg. In some embodiments, the muscarinic receptor antagonist is administered at a dosage of from about 2.5 to about 7.5 mg. In some embodiments, the composition is for use in treating a subject having a condition associated with pharyngeal airway collapse. In some embodiments, the condition associated with pharyngeal airway collapse is Sleep Apnea or Simple Snoring. In some embodiments, the condition associated with pharyngeal airway collapse is Obstructive Sleep Apnea (OSA). In some embodiments, the subject is in a non-fully conscious state. In some embodiments, the non-fully conscious state is sleep.

Another aspect of the invention provides a norepinephrine reuptake inhibitor (NRI), a muscarinic receptor antagonist, and a carbonic anhydrase inhibitor for use in treating a subject having a condition associated with pharyngeal airway collapse.

Other aspects of the invention provides a kit comprising a norepinephrine reuptake inhibitor (NRI), a muscarinic receptor antagonist, and a carbonic anhydrase inhibitor. In some embodiments, the kit is for use in treating a subject having a condition associated with pharyngeal airway collapse.

Another aspects of the invention provides a method of treating a subject having a condition associated with pharyngeal airway collapse, the method comprising administering to a subject in need thereof an effective amount of (i) Atomoxetine; (ii) Oxybutynin; and (iii) Acetazolamide. In some embodiments, the Oxybutynin is substantially pure (R)-oxybutynin.

Other aspects of the invention provides a pharmaceutical composition comprising (i) Atomoxetine; (ii) Oxybutynin; and (iii) Acetazolamide, in a pharmaceutically acceptable carrier. In some embodiments, the Oxybutynin is substantially pure (R)-oxybutynin.

Another aspect of the invention provides a method for lowering loop gain and increasing muscle responsiveness in a subject having obstructive sleep apnea (OSA), the method comprising administering to a subject in need thereof an effective amount of (i) a norepinephrine reuptake inhibitor (NRI); (ii) a muscarinic receptor antagonist; and (iii) a carbonic anhydrase inhibitor. In some embodiments, the carbonic anhydrase inhibitor is selected from the group consisting of acetazolamide, dichlorophenamide, dorzolamide, brinzolamide, methazolamide, zonisamide, ethoxzolamide, topiramate, sultiame, and any combinations thereof. In some embodiments, the carbonic anhydrase inhibitor is acetazolamide. In some embodiments, the NRI is selected from the group consisting of Atomoxetine and Reboxetine. In some embodiments, the NRI is Atomoxetine. In some embodiments, the muscarinic receptor antagonist comprises oxybutynin. In some embodiments, the oxybutynin is a substantially pure (R)-oxybutynin. In some embodiments, the oxybutynin is a racemic mixture of (R)-oxybutynin and (S)-oxybutynin. In some embodiments, the condition associated with pharyngeal airway collapse is Sleep Apnea or Simple Snoring. In some embodiments, the condition associated with pharyngeal airway collapse is Obstructive Sleep Apnea (OSA). In some embodiments, the subject is in a non-fully conscious state. In some embodiments, the non-fully conscious state is sleep. In some embodiments, the NRI, muscarinic receptor antagonist, and carbonic anhydrase inhibitor are administered in a single composition. In some embodiments, the single composition is an oral administration form. In some embodiments, the oral administration form is a syrup, pill, tablet, troche, capsule, or patch.

Another aspect of the invention provides a method for lowering loop gain and increasing muscle responsiveness in a subject having obesity hypoventilation syndrome (OHS), the method comprising administering to a subject in need thereof an effective amount of (i) a norepinephrine reuptake inhibitor (NRI); (ii) a muscarinic receptor antagonist; and (iii) a carbonic anhydrase inhibitor. In some embodiments, the carbonic anhydrase inhibitor is selected from the group consisting of acetazolamide, dichlorophenamide, dorzolamide, brinzolamide, methazolamide, zonisamide, ethoxzolamide, topiramate, sultiame, and any combinations thereof. In some embodiments, the carbonic anhydrase inhibitor is acetazolamide. In some embodiments, the NRI is selected from the group consisting of Atomoxetine and Reboxetine. In some embodiments, the NRI is Atomoxetine. In some embodiments, the muscarinic receptor antagonist comprises oxybutynin. In some embodiments, the oxybutynin is a substantially pure (R)-oxybutynin. In some embodiments, the oxybutynin is a racemic mixture of (R)-oxybutynin and (S)-oxybutynin. In some embodiments, the NRI, muscarinic receptor antagonist, and carbonic anhydrase inhibitor are administered in a single composition. In some embodiments, the single composition is an oral administration form. In some embodiments, the oral administration form is a syrup, pill, tablet, troche, capsule, or patch.

Unless otherwise defined, all technical and scientific terms used herein have the same meaning as commonly understood by one of ordinary skill in the art to which this invention belongs. Methods and materials are described herein for use in the present invention; other suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### BRIEF DESCRIPTION OF THE DRAWINGS

The following figures are provided by way of example and are not intended to limit the scope of the claimed invention.

FIG. 1. Graphic illustration of an obstructive apnea. The top channel shows the electroencephalogram (EEG) pattern of sleep. The next channel represents airflow. The next three channels show ventilatory effort by movements of the rib cage and abdomen and changes in esophageal pressure, all of which reflect contraction of respiratory muscles. The last channel indicates oxyhemoglobin saturation.

FIG. 2. A study scheme illustration outlining the study design randomizing patients into four sequences including blocks of four with respect to dosing schedules.

### DETAILED DESCRIPTION

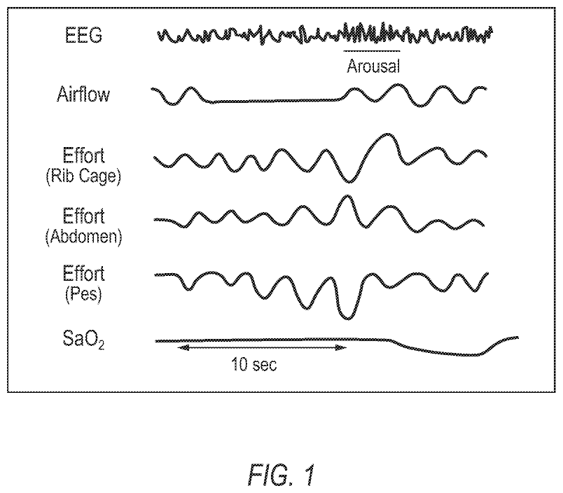

In humans, the pharyngeal airway region has no bone or cartilage support, and it is held open by muscles. When these muscles relax during sleep, the pharynx can collapse resulting in cessation of airflow. As shown in FIG. 1, ventilatory effort continues and increases in an attempt to overcome the obstruction, shown by an increase in esophageal pressure change. Rib cage and abdominal movements are in the opposite direction as a result of the diaphragm contracting against an occluded airway, forcing the abdominal wall to distend out and the chest wall to cave inward.

Increasing efforts to breathe lead to an arousal from sleep and result in opening of the airway and a resumption of normal breathing which can be monitored on an EEG (FIG. 1). The lack of airflow during the apnea also causes hypoxia, shown by a drop in oxyhemoglobin saturation (FIG. 1). Severity is generally measured using the apnea-hypopnea index (AHI), which is the combined average number of apneas (cessation of breathing for at least ten seconds) and hypopneas (reduced airflow and oxygen saturation) that occur per hour of sleep.

Obstructive sleep apnea (OSA) is characterized by repetitive collapse or ‘obstruction’ of the pharyngeal airway during sleep. When a stringent definition of OSA is used (an AHI of >15 events per hour or AHI >5 events per hour with daytime sleepiness), the estimated prevalence is approximately 15 percent in males and 5 percent in females. An estimated 30 million individuals in the United States have OSA, of which approximately 6 million have been diagnosed. The prevalence of OSA in the United States appears to be increasing due to aging and increasing rates of obesity. These obstructions (hypopneas/apneas) cause intermittent arousal from sleep and recurrent hypoxia, often manifesting themselves in the subject as excessive daytime sleepiness and decreased quality of life. OSA is associated with major comorbidities and economic costs, including: hypertension, diabetes, cardiovascular disease, motor vehicle accidents, workplace accidents, and fatigue/lost productivity.

Current evidence suggests that OSA pathogenesis involves the interactions of at least four physiological traits comprising: i) the pharyngeal anatomy and its propensity towards collapse; ii) the ability of the upper airway dilator muscles to activate and reopen the airway during sleep (i.e. neuromuscular compensation); iii) the arousal threshold from sleep (i.e. the propensity for hypopneas/apneas to lead to arousal and fragmented sleep); and iv) the stability of the ventilatory feedback loop (i.e. loop gain). Continuous positive airway pressure (CPAP) is the most common treatment for OSA but it is often poorly tolerated and only ˜50% of patients diagnosed with OSA continue therapy beyond 3 months. CPAP, when used alone, rarely abolishes OSA completely, likely because correcting just one trait ignores the multifactorial cause and nature of OSA pathogenesis. As evidenced herein, a pharmaceutical approach that targets two or more of these four physiological traits using a multi-factorial approach is expected to provide a treatment with an efficacy that will approach or exceed that of CPAP treatment for many patients.

Several pharmacological studies have shown promise for improving OSA through targeting certain OSA traits including combinations of a noradrenergic agent (atomoxetine 80 mg) and antimuscarinic agent (oxybutynin 5 mg), referred to as “ato-oxy”, administered before sleep to improve upper airway muscle responsiveness and reduce OSA severity (apnea-hypopnea index, AHI) by ˜70% on a single night. Atomoxetine is a selective norepinephrine reuptake inhibitor and is the first non-stimulant drug approved for attention deficit hyperactivity disorder in children and adults; oxybutynin is a muscarinic antagonist approved for overactive bladder in adults and children. Repeated-dose administration of ato-oxy over a week (N=6) illustrated that the effect of the agents on OSA severity was maintained. The agents administered separately had minimal effect on OSA severity (N=9). All side effects observed were expected based on the known safety profiles of the separate agents; all resolved spontaneously and none were severe.

Although effective in reducing OSA severity, the ato-oxy combination may not completely resolve OSA. Residual events after anatomical interventions can arise as an effect of elevated loop gain. One avenue for improving the efficacy of the combination drug therapies for OSA is to concurrently lower “loop gain”. The research provided herein demonstrates that the administration of acetazolamide, a potent carbonic anhydrase inhibitor, reduces loop gain and OSA severity (AHI) by nearly 50%. Acetazolamide is a respiratory stimulant used in healthy adults at high-altitude (where it augments and stabilizes respiration) and has additionally been administered safely long-term for glaucoma and heart failure. In one embodiment, simultaneously lowering loop gain and increasing muscle responsiveness (i.e. treating both obstructive and central nature of sleep apnea) resolves OSA.

The combination therapies disclosed herein using a norepinephrine reuptake inhibitor (NRI), a muscarinic receptor antagonist, and a carbonic anhydrase inhibitor utilize a multi-factorial approach targeting two or more physiological phenotypic traits that reduce OSA severity as measured by the apnea-hypopnea index [AHI] and the arousal index.

Methods of Treatment

The methods described herein include methods for the treatment of disorders associated with pharyngeal airway muscle collapse during sleep. In some embodiments, the disorder is Obstructive Sleep Apnea (OSA) or Simple Snoring. The methods herein additionally include methods for the treatment of disorders where severely overweight people fail to breathe rapidly or deep enough resulting in low oxygen levels and high blood carbon dioxide levels. In some embodiments, the disorder is obesity hypoventilation syndrome (OHS) or Pickwickian syndrome. In some embodiments, the methods described herein can be used to treat both Obstructive Sleep Apnea (OSA) and obesity hypoventilation syndrome (OHS). Generally, the methods include administering a therapeutically effective amount of a norepinephrine reuptake inhibitor (NRI), a muscarinic receptor antagonist (MRA), and a carbonic anhydrase inhibitor (CAI) as described herein, to a subject who is in need of, or who has been determined to be in need of, such treatment. These methods of administering therapeutic combinations of atomoxetine (NRI), oxybutynin (MRA), and acetazolamide (CAI) additionally provide a means for simultaneously lowering loop gain and increasing muscle responsiveness in treating OSA.

As used in this context, to “treat” means to ameliorate at least one symptom of the disorder associated with pharyngeal airway collapse. Often, pharyngeal airway collapse during sleep results in snoring and/or an interruption in breathing (apnea or hypopnea), arousal from sleep, and reduced oxygenation (hypoxemia); thus, a treatment can result in a reduction in snoring, apneas/hypopneas, sleep fragmentation, and hypoxemia. Administration of a therapeutically effective amount of a compound described herein for the treatment of a subject with OSA will result in decreased AHI.

An effective amount can be administered in one or more administrations, applications or dosages. The compositions can be administered from one or more times per day to one or more times per week; including once every other day. In some embodiments, the compositions are administered daily. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments.

Dosage, toxicity and therapeutic efficacy of the therapeutic compounds (i.e., NRI, MRA, and CAI, in a single composition or in separate compositions) can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50.

The data obtained from cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma can be measured, for example, by high performance liquid chromatography.

In some embodiments, the methods include administering a dose of from about 20 mg to about 150 mg NRI, from about 1 mg to about 25 mg MRA, and from about 250 mg to about 750 mg carbonic anhydrase inhibitor. In other embodiments, the methods include administering from about 50 mg to about 100 mg NRI, from about 1 mg to about 15 mg MRA, and from about 250 mg to about 750 mg carbonic anhydrase inhibitor. In still other embodiments, the methods include administering either combined or separate dosages of 80 mg atomoxetine/5 mg (R)-oxybutynin/500 mg acetazolamide; 80 mg atomoxetine/5 mg oxybutynin/500 mg acetazolamide; 100 mg atomoxetine/5 mg (R)-oxybutynin/500 mg acetazolamide; 100 mg atomoxetine/5 mg oxybutynin/750 mg acetazolamide; or 80 mg atomoxetine/5 mg (R)-oxybutynin/750 mg acetazolamide, e.g., 15-60, e.g., 15-25, 20-30, or 20-45 minutes before sleep time.

Pharmaceutical Compositions and Methods of Administration

The methods described herein include the use of pharmaceutical compositions comprising a norepinephrine reuptake inhibitor, a muscarinic receptor antagonist, and a carbonic anhydrase inhibitor as active ingredients. These norepinephrine reuptake inhibitor, muscarinic receptor antagonist, and carbonic anhydrase inhibitor agents can be administered in a single composition or in separate compositions.

Exemplary norepinephrine reuptake inhibitors (NRIs) include the selective NRIs Amedalin (UK-3540-1), Atomoxetine (Strattera), CP-39,332, Daledalin (UK-3557-15), Edivoxetine (LY-2216684), Esreboxetine, Lortalamine (LM-1404), Nisoxetine (LY-94,939), Reboxetine (Edronax, Vestra), Talopram (Lu 3-010), Talsupram (Lu 5-005), Tandamine (AY-23,946), Viloxazine (Vivalan); non-selective NRIs include Amitriptiline, Amoxapine, Bupropion, Ciclazindol, Desipramine, Desvenlafaxine, Dexmethilphenidate, Diethylpropion, Doxepin, Duloxetine, Imipramine, Levomilnacipran, Manifaxine (GW-320,659), Maprotiline, Methylphenidate, Milnacipran, Nefazodone, Nortriptyline, Phendimetrazine, Phenmetrazine, Protryptyline, Radafaxine (GW-353,162), Tapentadol (Nucynta), Teniloxazine (Lucelan, Metatone) and Venlafaxine.

In some embodiments, the norepinephrine reuptake inhibitor is Atomoxetine.

Oxybutynin is an antimuscarinic drug and a muscarinic receptor antagonist. In some embodiments, the oxybutynin is a racemic mixture of (R)-oxybutynin and (S)-oxybutynin where the enantiomers are present in about equal stoichiometric amounts. In some embodiments, the (R)-oxybutynin and/or (S)-oxybutynin may be in a free base or salt form, e.g., hydrochloride salt. A composition comprising a mixture of oxybutynin enantiomers, as described herein, may further comprise an enantiomeric excess of (R)-oxybutynin relative to its enantiomeric pair (i.e., (S)-oxybutynin). The enantiomeric excess of (R)-oxybutynin in these mixtures may be ≥10%, ≥20%, ≥25%, ≥30%, ≥40%, ≥50%, ≥60%, ≥70%, ≥75%, ≥80%, or ≥90%.

In some embodiments, the muscarinic receptor antagonist is a substantially enantiomerically pure (R)-oxybutynin. In some embodiments, the (R)-oxybutynin may be in a free base or salt form, e.g., hydrochloride salt. A composition comprising substantially enantiomerically pure (R)-oxybutynin, as described herein, may have an enantiomeric excess of the substantially enantiomerically pure (R)-oxybutynin of ≥80%, ≥90%, ≥95%, ≥98%, ≥99%, ≥99.5%, ≥99.8% or ≥99.9%.

The carbonic anhydrase inhibitor may be selected from the group consisting of acetazolamide, dichlorophenamide, dorzolamide, brinzolamide, methazolamide, zonisamide, ethoxzolamide, topiramate, sultiame, and any combinations thereof. In some embodiments, the carbonic anhydrase inhibitor is acetazolamide.

Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration.

In some embodiments, the methods include administering a dose of from about 20 mg to about 150 mg atomoxetine (or a dose equivalent thereof of another NRI), from about 20 mg to about 100 mg atomoxetine, from about 50 mg to about 100 mg atomoxetine, or from about 75 mg to about 100 mg atomoxetine. In some embodiments, the methods include administering a dose of from about 0.1 mg to about 25 mg oxybutynin ((R)-oxybutynin or a dose equivalent thereof of another MRA), from about 1 mg to about 20 mg oxybutynin, from about 1 mg to about 10 mg oxybutynin, or from about 2.5 mg to about 7.5 mg oxybutynin. In some embodiments, the methods include administering a dose of from about 50 mg to about 1000 mg acetazolamide (or a dose equivalent thereof of another CAI), from about 100 mg to about 800 mg acetazolamide, from about 250 mg to about 750 mg acetazolamide, from about 500 mg to about 750 mg acetazolamide, or from about 450 mg to about 650 mg acetazolamide. In some embodiments, the norepinephrine reuptake inhibitor, muscarinic receptor antagonist, and carbonic anhydrase inhibitor agent are administered in a single composition, for example, an oral administration in a syrup, pill, tablet, capsule, or patch form.

Pharmaceutical compositions are typically formulated to be compatible with its intended route of administration. Examples of routes of administration include systemic oral or transdermal administration.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, N.Y.). For example, oral compositions generally include an inert diluent or an edible carrier. For the purpose of oral therapeutic administration, the active compound(s) can be incorporated with excipients and used in the form of pills, tablets, troches, or capsules, e.g., gelatin capsules. Oral compositions can also be prepared using a fluid carrier. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

Systemic administration of one or both of the compounds as described herein (i.e., one or both of a norepinephrine reuptake inhibitor and substantially enantiomerically pure (R)-oxybutynin) can also be by transdermal means, e.g., using a patch, gel, or lotion, to be applied to the skin. For transdermal administration, penetrants appropriate to the permeation of the epidermal barrier can be used in the formulation. Such penetrants are generally known in the art. For example, for transdermal administration, the active compounds can formulated into ointments, salves, gels, or creams as generally known in the art. The gel and/or lotion can be provided in individual sachets, or via a metered-dose pump that is applied daily; see, e.g., Cohn et al., Ther Adv Urol. 2016 April; 8(2): 83-90.

In one embodiment, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques, or obtained commercially, e.g., from Alza Corporation and Nova Pharmaceuticals, Inc. Liposomal suspensions can also be used as pharmaceutically acceptable carriers. These can be prepared according to methods known to those skilled in the art, for example, as described in U.S. Pat. No. 4,522,811.

The pharmaceutical compositions can be included in a container, pack, or dispenser together with instructions for administration or use in a method described herein.

### EXAMPLES

The invention is further described in the following examples, which do not limit the scope of the invention described in the claims. The effects of the NRI, MRA, and CAI pharmaceutical combinations on the physiological traits responsible for OSA can be monitored using polysomnography. For example, polysomnography can be used to detect: pharyngeal anatomy and its propensity towards collapse; the ability of the upper airway dilator muscles to activate and reopen the airway during sleep (i.e. neuromuscular compensation); a subjects arousal threshold from sleep (i.e. the propensity for hypopneas/apneas to lead to arousal and fragmented sleep); and stability of the ventilatory control system feedback loop (i.e. loop gain). Each of these physiological traits are monitored and measured for changes with respect to the various dosing schedules outlined below. Baseline traits will be used to examine whether patient characteristics influence the responses to each combination of interventions (i.e. muscles, muscles plus loop gain, muscles plus arousal threshold)

### Example 1

### Study Procedures

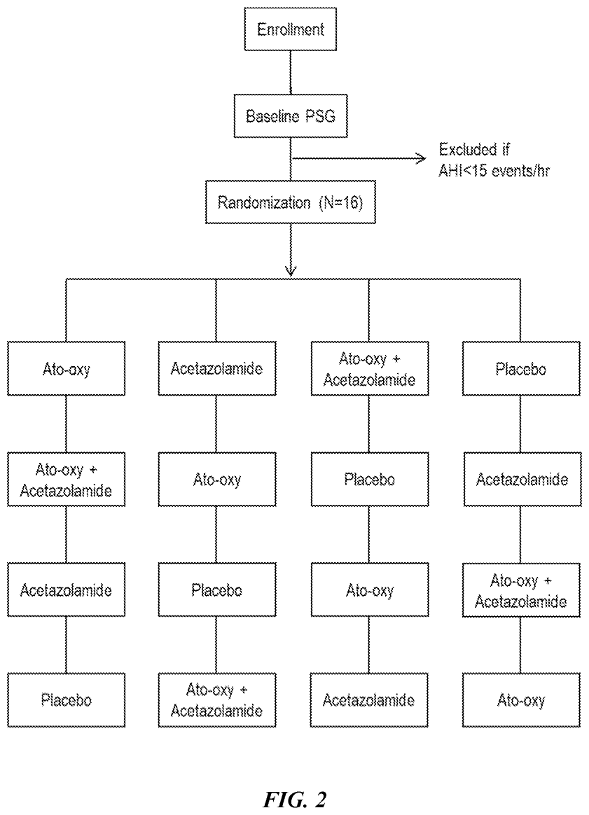

A placebo-controlled, double-blinded, randomized, crossover trial in OSA human patients is performed. Referring to the study schematic illustrated in FIG. 2, participants receive a randomized treatment order consisting of: 1) atomoxetine 80 mg+oxybutynin 5 mg; 2) atomoxetine 80 mg+oxybutynin 5 mg+acetazolamide 500 mg; 3) acetazolamide 500 mg; and 4) placebo, dosed 30 minutes before sleep. The combination of atomoxetine and oxybutynin is expected to reduce the apnea hypopnea index and all patients are expected to experience an improvement in OSA severity. The combination of Atomoxetine, oxybutynin, and acetazolamide is expected to reduce the apnea hypopnea index and reduce loop gain causing all patients to experience an improvement in OSA severity as compared to the placebo, acetazolamide, and atomoxetine/oxybutynin therapies. Additional benefits expected are increased genioglossus muscle responsiveness to an increase in ventilatory drive, improved upper airway muscle activity, improved ventilation, increased oxygen levels (SaO₂), increased total sleep time, and improved sleep efficiency.

### Study Preparation & Participant Screening

Participants will attend a screening to determine eligibility and consent to the study. The study will be explained in detail and an assessment will be made by an investigator to ensure the study procedures can be performed (including ability to tolerate wearing an oronasal mask) and written consent will be obtained. Subjects will complete baseline questionnaires, Epworth Sleepiness Scale, Functional Outcome of Sleep Questionnaire, plus four Visual Analog Scales (Sleep Quality, Excessive Fatigue, Waking Unrefreshed, Low Energy)

### Baseline Overnight Screening Sleep Study

A baseline overnight sleep study (see Measurements and Equipment) will also be performed to assess eligibility. The baseline study will used to determine if the subject has an apnea-hypopnea Index (AHI) greater than 15 events per hour. If their AHI is found to be less than 15 on the baseline visit, they will not be randomized and will discontinue participation. If the patient has performed a polysomnogram in the last 3 months at our laboratory under similar conditions (same equipment, see below) the baseline screening visit will be skipped.

### Treatment Nights

After consent and baseline visits, four overnight sleep studies will be performed approximately 1 week apart. The four sleep studies will consist of the following medication compositions: 1) atomoxetine 80 mg+oxybutynin 5 mg; 2) atomoxetine 80 mg+oxybutynin 5 mg+acetazolamide 500 mg; 3) acetazolamide 500 mg; or 4) placebo. At the conclusion of the baseline visit, the subject will be sent home with the corresponding medication for 3 days. The first day the patients will take half of the dose of the drugs (run-in period) to reduce the possible side effects. The medications will be prescribed for 3 nights instead of a single night to avoid the ‘first night effect’ on sleep and to collect more informed opinions from patients on their self-reported sleep quality while taking the agents at home in a familiar environment. The patients will perform these overnight sleep studies with the four difference test medications in random order.

Half doses will be given on the first night. Placebos capsules will be given such that there are no differences in number or appearance of capsules taken between periods A-D. After 2 days at home, on the third day of treatment the patient will arrive at the hospital and will perform a sleep study after taking the drugs or placebo. As the 5 overnight sleep studies will be performed approximately 1 week apart, there will be 4 days of washout period between consecutive different drugs combinations.

### Measurements and Equipment

Patients are untreated (e.g., no use of CPAP and/or oral appliance) as defined by absence of treatment for at least 1 week prior to the baseline study, with no plans to commence such treatment during the duration of the study.

Subjects will be instrumented with standard polysomnography (PSG) recording sensors. Sleep stage and arousals will be measured with electrodes pasted on to the scalp, face, chin and chest (EEG, EOG, EKG, chin EMG). Paste-on EMG electrodes will be placed over the anterior tibialis muscle to detect leg movements. Respiratory effort belts will be placed around the chest and abdomen to measure breathing movements. Oxygen saturation will be measured continuously with a pulse oximetry probe placed on either the fingertip or earlobe. Snoring will be detected with a small microphone positioned over the suprasternal notch. Body position will be recorded with a sensor taped to the thoracic belt. Each of these devices is standard for diagnostic PSG and should not be uncomfortable.

Oronasal flow will be monitored with a pneumotach and a pressure transducer attached to a sealed mask over the nose and held in place with straps. The mask allows monitoring of breathing (inspiratory flow by pneumotachograph which can be integrated to tidal volume) and expired carbon dioxide levels (PCO₂) using a calibrated infrared CO₂ analyzer (Capnograph/Oximeter Monitor), and if available, end-tidal oxygen levels using a calibrated O₂ analyzer.

On treatment nights, once all the equipment has been secured, subjects will be given their medication to take (30 min before lights out). Subjects will be allowed to fall asleep and data recording will begin. Subjects will be asked to remain in the supine position for 50-to-75% percent of the night. The study will end at approximately 6:00-6:30 am, at which time the monitoring equipment will be removed.

The morning after the sleep study, supine blood pressure will be measured (triplicate), equipment will be removed, and side effects will be assessed systematically from a list of the possible side effects of all drugs used in the trial. Five Visual Analog Scales will be performed (Sleep Quality, Excessive Fatigue, Waking Unrefreshed, Low Energy, and Treatment Satisfaction), and subjects will also be asked if they would continue taking the medication if given the opportunity.

An Emerald device will be attached to the wall by the bed for the overnight sleep studies done at BWH. This device will be used as a non-contact method to record physiological measures of ventilation during sleep. The device sits on the wall near the bed and analyzes how wireless signals reflect off the human body. This single device, much like a Wi-Fi router, does not require the subject to wear any sensors on their body, and operates across walls and most other obstructions. The ventilation signals collected by the device will be compared to both our polysomnographic and EEG signals.

If the data collected are considered insufficient, the subject may be asked to repeat the whole study or a part of it.

### Data Analysis

Apneas, hypopneas, sleep stages and arousals from sleep will be scored using current AASM guidelines (hypopneas defined by at least a 30% reduction in airflow in conjunction with either 3% desaturation or arousal) by a technician blinded to the study condition.

Phenotypic traits (collapsibility, responsiveness, arousal threshold, loop gain) values will be calculated by an investigator blinded to the randomization order. In brief, these traits will be measured using scored sleep study data, with a focus on the ventilatory flow signal. First the sleep study is segmented into 7-min overlapping windows containing non-REM sleep. A “ventilatory drive” signal (the level of ventilation that would be observed if the airway was open, akin to neural output to the diaphragm muscle) is estimated using measured ventilation data (tidal volume×respiratory rate) and a chemoreflex model fit to ventilation data when the airway is considered open (between scored respiratory events). The model parameters are used to describe the chemoreflex “loop gain”, namely the magnitude of the ventilatory drive increase in response to a prior reduction in ventilation. The arousal threshold is calculated as the value of ventilatory drive on the breath preceding each scored EEG arousal from sleep. Collapsibility is taken as the value of ventilation (sleep only) at normal ventilatory drive. Compensation is taken as the increase in ventilation that is achieved between the value at normal drive to that at maximal drive (at the arousal threshold). Parameters have been validated previously. Median overnight values are used to represent each individual patient per treatment period.

### REFERENCES

1. Strollo, P. J., Jr., et al., Upper-airway stimulation for obstructive sleep apnea. N Engl J Med, 2014. 370(2): p. 139-49.

2. Phillips, C. L., et al., Health outcomes of continuous positive airway pressure versus oral appliance treatment for obstructive sleep apnea: a randomized controlled trial. Am J Respir Crit Care Med, 2013. 187(8): p. 879-87.

3. Kushida, C. A., et al., Effects of continuous positive airway pressure on neurocognitive function in obstructive sleep apnea patients: The Apnea Positive Pressure Long-term Efficacy Study (APPLES). Sleep, 2012. 35(12): p. 1593-602.

4. Taylor, K. S., et al., Arousal From Sleep and Sympathetic Excitation During Wakefulness. Hypertension, 2016. 68(6): p. 1467-1474.

5. Azarbarzin, A., et al., The hypoxic burden of sleep apnoea predicts cardiovascular disease-related mortality: the Osteoporotic Fractures in Men Study and the Sleep Heart Health Study. Eur Heart J, 2018.

6. Wellman, A., et al., A method for measuring and modeling the physiological traits causing obstructive sleep apnea. J Appl Physiol, 2011. 110(6): p. 1627-37.

7. White, D. P., Pathogenesis of obstructive and central sleep apnea. Am J Respir Crit Care Med, 2005. 172(11): p. 1363-70.

8. Sands, S. A., et al., Enhanced Upper-airway Muscle Responsiveness is a Distinct Feature of Overweight/Obese Individuals Without Sleep Apnea. Am J Respir Crit Care Med, 2014.

9. Sands, S. A., et al., Identifying obstructive sleep apnoea patients responsive to supplemental oxygen therapy. Eur Respir J, 2018. 52(3).

10. Ratnavadivel, R., et al., Marked reduction in obstructive sleep apnea severity in slow wave sleep. J Clin Sleep Med, 2009. 5(6): p. 519-24.

11. Ratnavadivel, R., et al., Upper airway function and arousability to ventilatory challenge in slow wave versus stage 2 sleep in obstructive sleep apnoea. Thorax, 2010. 65(2): p. 107-12.

12. Younes, M., Role of arousals in the pathogenesis of obstructive sleep apnea. Am J Respir Crit Care Med, 2004. 169(5): p. 623-33.

13. Younes, M., Role of respiratory control mechanisms in the pathogenesis of obstructive sleep disorders. J Appl Physiol, 2008. 105(5): p. 1389-405.

14. Eckert, D. J., et al., Eszopiclone increases the respiratory arousal threshold and lowers the apnoea/hypopnoea index in obstructive sleep apnoea patients with a low arousal threshold. Clin Sci (Lond), 2011. 120(12): p. 505-14.

15. Eckert, D. J., et al., Defining phenotypic causes of obstructive sleep apnea. Identification of novel therapeutic targets. Am J Respir Crit Care Med, 2013. 188(8): p. 996-1004.

16. Edwards, B. A., et al., Upper-Airway Collapsibility and Loop Gain Predict the Response to Oral Appliance Therapy in Patients with Obstructive Sleep Apnea. Am J Respir Crit Care Med, 2016. 194(11): p. 1413-1422.

17. Edwards, B. A., et al., Acetazolamide improves loop gain but not the other physiological traits causing obstructive sleep apnoea. J Physiol, 2012. 590(Pt 5): p. 1199-211.

18. Edwards, B. A., et al., The Combination of Supplemental Oxygen and a Hypnotic Markedly Improves Obstructive Sleep Apnea in Patients with a Mild to Moderate Upper Airway Collapsibility. Sleep, 2016. 39(11): p. 1973-1983.

19. Kribbs, N. B., et al., Objective measurement of patterns of nasal CPAP use by patients with obstructive sleep apnea. Am Rev Respir Dis, 1993. 147(4): p. 887-95.

20. Engleman, H. M. and M. R. Wild, Improving CPAP use by patients with the sleep apnoea/hypopnoea syndrome (SAHS). Sleep Med Rev, 2003. 7(1): p. 81-99.

21. Heinzer, R. C., et al., Trazodone increases arousal threshold in obstructive sleep apnoea. Eur Respir J, 2008. 31(6): p. 1308-12.

22. Taranto-Montemurro, L., et al., The Combination of Atomoxetine and Oxybutynin Greatly Reduces Obstructive Sleep Apnea Severity: A Randomized, Placebo-Controlled, Double-Blind Crossover Trial. Am J Respir Crit Care Med, 2018.

23. Witcher, J. W., et al., Atomoxetine pharmacokinetics in children and adolescents with attention deficit hyperactivity disorder. J Child Adolesc Psychopharmacol, 2003. 13(1): p. 53-63.

24. Sauer, J. M., B. J. Ring, and J. W. Witcher, Clinical pharmacokinetics of atomoxetine. Clin Pharmacokinet, 2005. 44(6): p. 571-90.

25. Wernicke, J. F., et al., Cardiovascular effects of atomoxetine in children, adolescents, and adults. Drug Saf, 2003. 26(10): p. 729-40.

26. Wernicke, J. F., et al., Changes in symptoms and adverse events after discontinuation of atomoxetine in children and adults with attention deficit/hyperactivity disorder: a prospective, placebo-controlled assessment. J Clin Psychopharmacol, 2004. 24(1): p. 30-5.

27. Biederman, J., et al., Efficacy of atomoxetine versus placebo in school-age girls with attention-deficit/hyperactivity disorder. Pediatrics, 2002. 110(6): p. e75.

28. Hughes, K. M., et al., Measurement of oxybutynin and its N-desethyl metabolite in plasma, and its application to pharmacokinetic studies in young, elderly and frail elderly volunteers. Xenobiotica, 1992. 22(7): p. 859-69.

29. Ouslander, J. G., et al., Pharmacokinetics and clinical effects of oxybutynin in geriatric patients. J Urol, 1988. 140(1): p. 47-50.

30. Yarker, Y. E., K. L. Goa, and A. Fitton, Oxybutynin. A review of its pharmacodynamic and pharmacokinetic properties, and its therapeutic use in detrusor instability. Drugs Aging, 1995. 6(3): p. 243-62.

31. Sommer, B. R., et al., The effect of oxybutynin treatment on cognition in children with diurnal incontinence. J Urol, 2005. 173(6): p. 2125-7.

32. Giramonti, K. M., B. A. Kogan, and L. F. Halpern, The effects of anticholinergic drugs on attention span and short-term memory skills in children. Neurourol Urodyn, 2008. 27(4): p. 315-8.

33. Gish, P., et al., Spectrum of central anticholinergic adverse effects associated with oxybutynin: comparison of pediatric and adult cases. J Pediatr, 2009. 155(3): p. 432-4.

34. Joosten, S. A., et al., Loop Gain Predicts the Response to Upper Airway Surgery in Patients With Obstructive Sleep Apnea. Sleep, 2017. 40(7).

35. Li, Y., et al., Physiology-Based Modeling May Predict Surgical Treatment Outcome for Obstructive Sleep Apnea. J Clin Sleep Med, 2017. 13(9): p. 1029-1037.

36. Fontana, M., et al., Effect of acetazolamide on chemosensitivity, Cheyne-Stokes respiration, and response to effort in patients with heart failure. Am J Cardiol, 2011. 107(11): p. 1675-80.

37. Nussbaumer-Ochsner, Y., et al., Patients with obstructive sleep apnea syndrome benefit from acetazolamide during an altitude sojourn: a randomized, placebo-controlled, double-blind trial. Chest, 2012. 141(1): p. 131-8.

38. Glidewell, R. N., W. C. Orr, and N. Imes, Acetazolamide as an adjunct to CPAP treatment: a case of complex sleep apnea in a patient on long-acting opioid therapy. J Clin Sleep Med, 2009. 5(1): p. 63-4.

39. Javaheri, S., Acetazolamide improves central sleep apnea in heart failure: a double-blind, prospective study. Am J Respir Crit Care Med, 2006. 173(2): p. 234-7.

40. Nunez, J., et al., Use of acetazolamide in the treatment of patients with refractory congestive heart failure. Cardiovasc Ther, 2018. 36(6): p. e12465.

41. Caravita, S., et al., Sex and acetazolamide effects on chemoreflex and periodic breathing during sleep at altitude. Chest, 2015. 147(1): p. 120-131.

42. Williamson, J., P. Oakeshott, and J. Dallimore, Altitude sickness and acetazolamide. BMJ, 2018. 361: p. k2153.

43. Burns, P., et al., Altitude Sickness Prevention with Ibuprofen Relative to Acetazolamide. Am J Med, 2019. 132(2): p. 247-251.

44. De Carvalho, C. A., C. Lawrence, and H. H. Stone, Acetazolamide (diamox) therapy in chronic glaucoma; a three-year follow-up study. AMA Arch Ophthalmol, 1958. 59(6): p. 840-9.

45. Jordan, A. S., et al., Physiology of Arousal in OSA and Potential Impacts for Sedative Treatment. Am J Respir Crit Care Med, 2017.

46. Loewen, A. H., et al., Response of genioglossus muscle to increasing chemical drive in sleeping obstructive apnea patients. Sleep, 2011. 34(8): p. 1061-73.

47. White, D. P. and M. K. Younes, Obstructive sleep apnea. Compr Physiol, 2012. 2(4): p. 2541-94.

48. Carberry, J. C., et al., Role of common hypnotics on the phenotypic causes of obstructive sleep apnoea: paradoxical effects of zolpidem. Eur Respir J, 2017. 50(6).

49. Monti, J. M., et al., Zolpidem and rebound insomnia—a double-blind, controlled polysomnographic study in chronic insomniac patients. Pharmacopsychiatry, 1994. 27(4): p. 166-75.

50. Ware, J. C., et al., Minimal rebound insomnia after treatment with 10-mg zolpidem. Clin Neuropharmacol, 1997. 20(2): p. 116-25.

51. Roehrs, T. A., et al., Twelve months of nightly zolpidem does not lead to rebound insomnia or withdrawal symptoms: a prospective placebo-controlled study. J Psychopharmacol, 2012. 26(8): p. 1088-95.

52. Berry, R. B. and P. B. Patel, Effect of zolpidem on the efficacy of continuous positive airway pressure as treatment for obstructive sleep apnea. Sleep, 2006. 29(8): p. 1052-6.

53. Quadri, S., C. Drake, and D. W. Hudgel, Improvement of idiopathic central sleep apnea with zolpidem. J Clin Sleep Med, 2009. 5(2): p. 122-9.

54. DeBacker, W. A., et al., Central apnea index decreases after prolonged treatment with acetazolamide. Am J Respir Crit Care Med, 1995. 151(1): p. 87-91.

55. White, D. P., et al., Central sleep apnea. Improvement with acetazolamide therapy. Arch Intern Med, 1982. 142(10): p. 1816-9.

56. Hazouard, E., et al., [Salicylism and glaucoma: reciprocal augmentation of the toxicity of acetazolamide and acetylsalicylic acid]. J Fr Ophtalmol, 1999. 22(1): p. 73-5.

57. Terrill, P. I., et al., Quantifying the ventilatory control contribution to sleep apnoea using polysomnography. Eur Respir J, 2015. 45(2): p. 408-18.

58. Sands, S. A., et al., Phenotyping Sleep Apnea Using Polysomnography: Upper Airway Collapsibility And Responsiveness [Abstract]. Am J Respir Crit Care Med, 2016. 193: p. A6378.

59. Sands, S. A., et al., Quantifying the Arousal Threshold Using Polysomnography in Obstructive Sleep Apnea. Sleep, 2018. 41(1).

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of treating a subject having a condition associated with pharyngeal airway collapse, the method comprising administering to a subject in need thereof an effective amount of (i) a norepinephrine reuptake inhibitor (NRI); (ii) a muscarinic receptor antagonist; and (iii) a carbonic anhydrase inhibitor.

2. The method of claim 1, wherein the carbonic anhydrase inhibitor is selected from the group consisting of acetazolamide, dichlorophenamide, dorzolamide, brinzolamide, methazolamide, zonisamide, ethoxzolamide, topiramate, sultiame, and any combination thereof.

3. The method of either of claim 1 or 2, wherein the carbonic anhydrase inhibitor is acetazolamide.

4. The method of any one of claims 1-3, wherein the NRI is selected from the group consisting of Atomoxetine and Reboxetine.

5. The method of claim 4, wherein the NRI is Atomoxetine.

6. The method of any one of claims 1-3, wherein the NRI is a norepinephrine selective reuptake inhibitor (NSRI).

7. The method of claim 6, wherein the NSRI is selected from the group consisting of Amedalin, Atomoxetine, CP-39,332, Daledalin, Edivoxetine, Esreboxetine, Lortalamine, Nisoxetine, Reboxetine, Talopram, Talsupram, Tandamine, and Viloxazine.

8. The method of any one of claims 1-3, wherein the NRI is a norepinephrine non-selective reuptake inhibitor (NNRI) selected from the group consisting of Amitriptiline, Amoxapine, Bupropion, Ciclazindol, Desipramine, Desvenlafaxine, Dexmethilphenidate, Diethylpropion, Doxepin, Duloxetine, Imipramine, Levomilnacipran, Manifaxine, Maprotiline, Methylphenidate, Milnacipran, Nefazodone, Nortriptyline, Phendimetrazine, Protryptyline, Radafaxine, Tapentadol, Teniloxazine, and Venlafaxine.

9. The method of any one of claims 1-8, wherein the muscarinic receptor antagonist comprises oxybutynin.

10. The method of claim 9, wherein the oxybutynin is a substantially pure (R)-oxybutynin.

11. The method of claim 9, wherein the oxybutynin is a racemic mixture of (R)-oxybutynin and (S)-oxybutynin.

12. The method of any one of claims 1-11, wherein the carbonic anhydrase inhibitor, such as acetazolamide, is administered at a dosage of from about 250 mg to about 750 mg.

13. The method of claim 12, wherein the carbonic anhydrase inhibitor, such as acetazolamide, is administered at a dosage of about 500 mg.

14. The method of any one of claims 1-13, wherein the NRI, such as atomoxetine, is administered at a dosage of from about 20 to about 150 mg.

15. The method of claim 14, wherein the NRI, such as atomoxetine, is administered at a dosage of from about 50 to about 100 mg.

16. The method of any one of claims 1-15, wherein the muscarinic receptor antagonist, such as oxybutynin, is administered at a dosage of from about 1 to about 15 mg.

17. The method of claim 16, wherein the muscarinic receptor antagonist, such as oxybutynin, is administered at a dosage of from about 2.5 to about 7.5 mg.

18. The method of any one of claims 1-17, wherein the condition associated with pharyngeal airway collapse is Sleep Apnea or Simple Snoring.

19. The method of claim 18, wherein the condition associated with pharyngeal airway collapse is Obstructive Sleep Apnea (OSA).

20. The method of any one of claims 1-19, wherein the subject is in a non-fully conscious state.

21. The method of claim 20, wherein the non-fully conscious state is sleep.

22. The method of any one of claims 1-21, wherein the NRI, muscarinic receptor antagonist, and carbonic anhydrase inhibitor are administered in a single composition.

23. The method of claim 22, wherein the single composition is an oral administration form.

24. The method of claim 23, wherein the oral administration form is a syrup, pill, tablet, troche, capsule, or patch.

25. A pharmaceutical composition comprising (i) a norepinephrine reuptake inhibitor (NRI); (ii) a muscarinic receptor antagonist; and (iii) a carbonic anhydrase inhibitor, in a pharmaceutically acceptable carrier.

26. The composition of claim 25, wherein the carbonic anhydrase inhibitor is selected from the group consisting of acetazolamide, dichlorophenamide, dorzolamide, brinzolamide, methazolamide, zonisamide, ethoxzolamide, topiramate, sultiame, and any combinations thereof.

27. The composition of either of claim 25 or 26, wherein the carbonic anhydrase inhibitor is acetazolamide.

28. The composition of any one of claims 25-27, wherein the NRI is selected from the group consisting of Atomoxetine and Reboxetine.

29. The composition of claim 28, wherein the NRI is Atomoxetine.

30. The composition of any one of claims 25-27, wherein the NRI is a norepinephrine selective reuptake inhibitor (NSRI).

31. The composition of claim 30, wherein the NSRI is selected from the group consisting of Amedalin, Atomoxetine, CP-39,332, Daledalin, Edivoxetine, Esreboxetine, Lortalamine, Nisoxetine, Reboxetine, Talopram, Talsupram, Tandamine, and Viloxazine.

32. The composition of any one of claims 25-27, wherein the NRI is a norepinephrine non-selective reuptake inhibitor (NNRI) selected from the group consisting of Amitriptiline, Amoxapine, Bupropion, Ciclazindol, Desipramine, Desvenlafaxine, Dexmethilphenidate, Diethylpropion, Doxepin, Duloxetine, Imipramine, Levomilnacipran, Manifaxine, Maprotiline, Methylphenidate, Milnacipran, Nefazodone, Nortriptyline, Phendimetrazine, Protryptyline, Radafaxine, Tapentadol, Teniloxazine, and Venlafaxine.

33. The composition of any one of claims 25-32, wherein the muscarinic receptor antagonist comprises oxybutynin.

34. The composition of claim 33, wherein the oxybutynin is a substantially pure (R)-oxybutynin.

35. The composition of claim 33, wherein the oxybutynin is a racemic mixture of (R)-oxybutynin and (S)-oxybutynin.

36. The composition of any one of claims 25-35, wherein the carbonic anhydrase inhibitor, such as acetazolamide, is present in an amount of from about 250 mg to about 750 mg.

37. The composition of claim 36, wherein the carbonic anhydrase inhibitor, such as acetazolamide, is present in an amount of about 500 mg.

38. The composition of any one of claims 25-37, wherein the NRI, such as atomoxetine, is present in an amount of from about 20 to about 150 mg.

39. The composition of claim 38, wherein the NRI, such as atomoxetine, is present in an amount of from about 50 to about 100 mg.

40. The composition of any one of claims 25-39, wherein the muscarinic receptor antagonist, such as oxybutynin, is present in an amount of from about 1 to about 15 mg.

41. The composition of claim 40, wherein the muscarinic receptor antagonist, such as oxybutynin, is present in an amount of from about 2.5 to about 7.5 mg.

42. The composition of any one of claims 25-41, wherein the pharmaceutical formulation is in an immediate release formulation.

43. The composition of any one of claims 25-41, wherein the pharmaceutical formulation is in an extended release formulation.

44. The composition of any one of claims 25-43, for use in treating a subject having a condition associated with pharyngeal airway collapse.

45. The composition for use of claim 44, wherein the condition associated with pharyngeal airway collapse is Sleep Apnea or Simple Snoring.

46. The composition for use of claim 45, wherein the condition associated with pharyngeal airway collapse is Obstructive Sleep Apnea (OSA).

47. The composition for use of any one of claims 44-46, wherein the subject is in a non-fully conscious state.

48. The composition for use of claim 47, wherein the non-fully conscious state is sleep.

49. A norepinephrine reuptake inhibitor (NRI), a muscarinic receptor antagonist, and a carbonic anhydrase inhibitor for use in treating a subject having a condition associated with pharyngeal airway collapse.

50. A kit comprising a norepinephrine reuptake inhibitor (NRI), a muscarinic receptor antagonist, and a carbonic anhydrase inhibitor.

51. The kit of claim 50, for use in treating a subject having a condition associated with pharyngeal airway collapse.

52. A method of treating a subject having a condition associated with pharyngeal airway collapse, the method comprising administering to a subject in need thereof an effective amount of (i) Atomoxetine; (ii) Oxybutynin; and (iii) Acetazolamide.

53. A pharmaceutical composition comprising (i) Atomoxetine; (ii) Oxybutynin; and (iii) Acetazolamide, in a pharmaceutically acceptable carrier.

54. A method for lowering loop gain and increasing muscle responsiveness in a subject having obstructive sleep apnea (OSA), the method comprising administering to a subject in need thereof an effective amount of (i) a norepinephrine reuptake inhibitor (NRI); (ii) a muscarinic receptor antagonist; and (iii) a carbonic anhydrase inhibitor.

55. The method of claim 54, wherein the carbonic anhydrase inhibitor is selected from the group consisting of acetazolamide, dichlorophenamide, dorzolamide, brinzolamide, methazolamide, zonisamide, ethoxzolamide, topiramate, sultiame, and any combinations thereof.

56. The method of either of claim 54 or 55, wherein the carbonic anhydrase inhibitor is acetazolamide.

57. The method of any one of claims 54-56, wherein the NRI is selected from the group consisting of Atomoxetine and Reboxetine.

58. The method of any one of claims 54-57, wherein the NRI is Atomoxetine.

59. The method of any one of claims 54-58, wherein the muscarinic receptor antagonist comprises oxybutynin.

60. The method of claim 59, wherein the oxybutynin is a substantially pure (R)-oxybutynin.

61. The method of claim 59, wherein the oxybutynin is a racemic mixture of (R)-oxybutynin and (S)-oxybutynin.

62. The method of any one of claims 54-61, wherein the condition associated with pharyngeal airway collapse is Sleep Apnea or Simple Snoring.

63. The method of claim 62, wherein the condition associated with pharyngeal airway collapse is Obstructive Sleep Apnea (OSA).

64. The method of any one of claims 54-63, wherein the subject is in a non-fully conscious state.

65. The method of claim 64, wherein the non-fully conscious state is sleep.

66. The method of any one of claims 54-65, wherein the NRI, muscarinic receptor antagonist, and carbonic anhydrase inhibitor are administered in a single composition.

67. The method of claim 66, wherein the single composition is an oral administration form.

68. The method of claim 67, wherein the oral administration form is a syrup, pill, tablet, troche, capsule, or patch.

69. A method for lowering loop gain and increasing muscle responsiveness in a subject having obesity hypoventilation syndrome (OHS), the method comprising administering to a subject in need thereof an effective amount of (i) a norepinephrine reuptake inhibitor (NRI); (ii) a muscarinic receptor antagonist; and (iii) a carbonic anhydrase inhibitor.

70. The method of claim 69, wherein the carbonic anhydrase inhibitor is selected from the group consisting of acetazolamide, dichlorophenamide, dorzolamide, brinzolamide, methazolamide, zonisamide, ethoxzolamide, topiramate, sultiame, and any combinations thereof.

71. The method of either of claim 69 or 70, wherein the carbonic anhydrase inhibitor is acetazolamide.

72. The method of any one of claims 69-71, wherein the NRI is selected from the group consisting of Atomoxetine and Reboxetine.

73. The method of any one of claims 69-72, wherein the NRI is Atomoxetine.

74. The method of any one of claims 69-73, wherein the muscarinic receptor antagonist comprises oxybutynin.

75. The method of claim 74, wherein the oxybutynin is a substantially pure (R)-oxybutynin.

76. The method of claim 74, wherein the oxybutynin is a racemic mixture of (R)-oxybutynin and (S)-oxybutynin.

77. The method of any one of claims 69-76, wherein the subject has both obesity hypoventilation syndrome (OHS) and obstructive sleep apnea (OSA).

78. The method of any one of claims 69-77, wherein the NRI, muscarinic receptor antagonist, and carbonic anhydrase inhibitor are administered in a single composition.

79. The method of claim 78, wherein the single composition is an oral administration form.

80. The method of claim 79, wherein the oral administration form is a syrup, pill, tablet, troche, capsule, or patch.
